(19) United States
(12) Patent Application Publication
(10) Pub. No.: US 2009/0081747 A1
(43) Pub. Date: Mar. 26, 2009

(54) ETHANOL FROM CORN AND USE OF DISTILLER GRAINS BY-PRODUCT TO PRODUCE FUEL GAS

(75) Inventor: Stanley R. Pearson, Baton Rouge, LA (US)

Correspondence Address: STANLEY R. PEARSON, 544 SOUTH LAKEVIEW DRIVE, BATON ROUGE, LA, 70810, US

(73) Assignee: International Financial Services #1, LLC, Baton Rouge, LA (US)

(21) Appl. No.: 12/237,984

(22) Filed: Sep. 25, 2008

Related U.S. Application Data
(60) Provisional application No. 60/995,192, filed on Sep. 25, 2007.

(51) Int. Cl. C12P 7/06

(52) U.S. Cl. 435/161

(57) ABSTRACT

A process for substantially increasing the production of ethanol from corn and other such biomass feedstocks. Ethanol, carbon dioxide and distiller grains are typically produced during the fermentation process. The carbon dioxide is used as a feed for additional ethanol make and the distiller grains can be used as a livestock feed or to produce a synthetic fuel gas that is used to run a boiler to produce steam used in the fermentation process.

### CROSS-REFERENCE TO RELATED APPLICATIONS

This application is based on Provisional Application U.S. Ser. No. 60/995,192 filed Sep. 25, 2007.

### FIELD OF THE INVENTION

The present invention relates to a process for substantially increasing the production of ethanol from corn and other such biomass feedstocks. Ethanol, carbon dioxide and distiller grains are typically produced during the fermentation process. The carbon dioxide is used as a feed for additional ethanol make and the distiller grains can be used as a livestock feed or to produce a synthetic fuel gas that is used to run a boiler to produce steam used in the fermentation process.

### BACKGROUND OF THE INVENTION

As the world's petroleum supplies continue to diminish there is a growing need for feedstocks that can be substituted for various petroleum products, particularly transportation fuels. One such substitute is bioethanol that is derived from renewal biomass materials, such as sugar and corn. In a conventional ethanol production process utilizing corn as the starch containing feedstock, the corn is ground to produce a milled corn call meal. The milling can be either dry milling or wet milling. The meal is then mixed with water and an enzyme, such as alpha-amylase, and passed to cookers where the starch is liquefied. Heat is applied at this stage to enhance liquefaction. Cookers with a high temperature stage of about 120 to 150° C. and a lower temperature holding period of about 95° C. are used. The high temperatures reduce bacteria levels in the resulting mash.

The mash from the cookers is cooled and a secondary enzyme such as glucoamylase, is added to convert the liquefied starch to fermentable sugars (dextrose). Yeast is added to the mash to ferment the sugars to ethanol and carbon dioxide. Using a continuous process, the fermenting mash is allowed to flow through several fermenters until it is fully fermented. In a batch process, the mash stays in a single fermenter for about 48 hours before distillation. The fermented mash, now called beer, contains about 10% alcohol plus all the non-fermentable solids from the corn and yeast cells. The mash is pumped to a distillation system where the alcohol is removed from the solids and the water. The alcohol leaves the distillation system at about 95% strength, and the residue mash, called stillage, is transferred from the distillation system to a co-product processing area. The two main co-products in the production of ethanol from a biomass such as corn is carbon dioxide and distillers grains.

The carbon dioxide is produced in relatively large quantities during fermentation and is typically vented to the atmosphere or sold to other industries. The distillers grains may or may not be suitable for use as a livestock feed. There are time when the distiller grains are contaminated with harmful microorganisms and thus will be of low value and not suitable as a livestock feed.

While commercial plants now exist for the production of ethanol from renewable sources, such as corn, they are not economical because of the large amounts of carbon dioxide and distillers grains that are produced and which are substantially lower in value than ethanol. The economics are further negatively affected when the distillers grains are unsuitable as a feed for livestock. Therefore, a need exists for processes from producing ethanol from biomass materials that can more economically utilize the large amounts of carbon dioxide and distillers grains produced in the process, especially in cases where the distiller grains have little of no value as a livestock feed.

### SUMMARY OF THE INVENTION

In accordance with the present invention there is provided a process for converting corn to ethanol, which process comprising:

a) milling corn to produce a powdered meal containing cornstarch;

b) adding an effective amount of water and a first enzyme to the meal to liquefy the corn starch thereby producing a mash;

c) cooking the mash for an effective amount of time at an effective temperatures in the presence of a second enzyme;

d) adding an effective amount of yeast to said cooked mash and passing said cooked mash containing said yeast to a fermentation zone;

e) fermenting said cooked mash containing said yeast for an effective amount of time thereby producing a first ethanol product stream, a carbon dioxide stream, and distiller grains;

f) distilling off the ethanol product;

g) collecting the ethanol product;

h) separating the carbon dioxide from the distiller grains;

i) drying said distiller grains;

j) determining if said distiller grains are suitable as a livestock feed;

k) collecting the distiller grains if the distiller grains are not suitable as a livestock feed;

l) conducting an effective amount of steam and at least a portion of the dried distiller grains to a mixing zone and forming a mixture to a steam reforming zone comprised of at least three serially connected reforming stages, each operated at progressively higher temperature and wherein the temperature of the reforming stage is from about 650° F. to about 800° F. and wherein a synthetic gaseous product stream is produced;

m) conducting an effective amount of a biomass co-feed to said mixing zone in the event the amount of distillers grains is insufficient for a predetermined feed rate to said reforming zone;

n) passing said synthetic gaseous product stream from said steam reforming zone to a heat recovery zone wherein the temperature of said synthetic gaseous product stream is substantially lowered and wherein steam is generated;

o) passing said lowered temperature synthetic gaseous product stream to a solids recovery zone wherein substantially any solids present are removed;

p) passing said synthetic gaseous product stream from said solids recovery zone to a organics removal zone wherein substantially any remaining organic material is removed by contact with an organic liquid in which the organic material is at least partially soluble;

q) passing said synthetic gaseous product stream from said organics removal zone to an acid gas removal zone wherein CO₂ is removed, thereby resulting in an acid gas depleted synthetic gaseous product stream and a CO₂ stream;

r) passing said acid gas depleted synthetic gaseous product stream as fuel to a boiler that produces steam;

s) passing at least a portion of said steam produced in s) above to the fermentation zone;

t) passing at least a portion of the carbon dioxide streams from both the fermentation zone and the acid gas removal zone, along with an effective amount of methane, to a catalytic steam reformer;

u) reforming said mixture of carbon dioxide and methane in the presence of a stream reforming catalyst at temperatures from about 800° C. to about 1000° C. thereby resulting in a synthesis gas product stream, which synthesis gas product stream is comprised of hydrogen, carbon oxides, and methane

v) passing said synthesis gas product stream to a first Fischer-Tropsch ethanol reactor wherein it is reacted in the presence of a catalyst capable of converting a portion of the synthesis gas to ethanol wherein there is produced a second ethanol product stream, steam, and a first purge gas stream comprised of unreacted synthesis gas;

w) passing said second ethanol product stream to a purification zone wherein impurities are removed form the ethanol;

x) passing said steam and purge gas stream from step v) to a second Fischer Tropsch ethanol reactor wherein a third ethanol product stream is produced steam, and a second purge gas stream comprised of unreacted synthesis gas;

y) passing said third ethanol product stream to a purification zone wherein impurities are removed;

z) passing at least a portion of said steam and second purge gas stream from step x) to said catalytic steam reformer.

### BRIEF DESCRIPTION OF THE FIGURES

FIG. 1 hereof is a schematic of a preferred four stage steam reformer of the present invention.

FIG. 2 hereof is a cross-sectional view, along plane A-A, of the stages 1 and 2 of the steam reformer of the present invention.

FIG. 3 hereof is a generalized flow scheme of a preferred embodiment of the present invention wherein corn is processed through a fermentation process to produce ethanol, carbon dioxide and distillers grains. The distillers grains are either collected or dried and steam reformed to produce a syngas, which is then sent through various clean-up steps then used as fuel for a boiler that produces heat for use in the fermentation zone. The carbon dioxide is combined with methane and reformed to produce a synthesis gas that is passed to a Fischer-Tropsch ethanol reactor where additional ethanol is produced.

### DETAILED DESCRIPTION OF THE INVENTION

Any suitable biomass material can used in the practice of the present invention as feed to the fermentation zone as long as it can economically produce ethanol, carbon dioxide and a solid biomass material, such as distillers grains that is capable of being used as a feed for livestock. Non-limiting examples of biomass materials suitable for use herein include corn and a sugar substance, such as blackstrap molasses. Preferred is corn. Any suitable process can be used to produce ethanol from corn. In a typical conventional process utilizing corn as the starch containing feedstock, the corn is ground to produce a milled corn call meal. The milling can be either dry milling or wet milling. The meal is then mixed with water and an enzyme such as alpha-amylase and then passed through cookers where the starch is liquefied. Heat is applied at this stage to enable liquefaction of the starch. Cookers having a high temperature stage of about 120 to 150° C. and a lower temperature holding period of about 95° C. are used. The high temperatures reduce bacteria levels in the resulting mash.

The mash from the cookers is cooled and a secondary enzyme (glucoamylase) is added to convert the liquefied starch to fermentable sugars (dextrose). Yeast is added to the mash to ferment the sugars to ethanol and carbon dioxide. Using a continuous process, the fermenting mash is allowed to flow through several fermenters until it is fully fermented and leaves the final tank. In a batch process, the mash stays in one fermenter for about 48 hours before the distillation process is started. The fermented mash, now called beer, contains about 10% alcohol plus all the non-fermentable solids from the corn and yeast cells. The mash is pumped to the distillation system where the alcohol is removed from the solids and the water. The alcohol leaves the distillation system at about 95% strength, and the residue mash, called stillage, is transferred from the distillation system to a co-product processing area. The two main co-products in the production of ethanol is carbon dioxide and distillers grains. Distillers grain, used wet or dry, can be a highly nutritious livestock feed, however it can also be contaminated with harmful microorganisms, typically bacteria, and not be of any value as livestock feed. The carbon dioxide is produced in relatively large quantities during fermentation and is typically either vented to the atmosphere or sold to other industries. Practice of the present invention uses this carbon dioxide to make additional ethanol and utilize contaminated distillers grains to produce a synthetic fuel gas that is used to fuel a boiler that produces heat for the overall process.

Generally, the reformer requires that a feedstock have less than about 15% moisture content, but there is an optimization between moisture content and conversion process efficiency. The actual moisture content will vary somewhat depending on the commercial process equipment used. Since some of the biomass received for processing can have a moisture content of from about 40 to 60% it will have to be dried before reforming. Any conventional drying technique can be used as long as the moisture content is lowered to less than about 15% when mixed with the superheated steam. For example, passive drying during summer storage can reduce the moisture content to about 30% or less. Active silo drying can reduce the moisture content down to about 12%. Drying can be accomplished either by very simple means, such as near ambient, solar drying or by waste heat flows or by specifically designed dryers operated on location. Also, commercial dryers are available in many forms and most common are rotary kilns and shallow fluidized bed dryers.

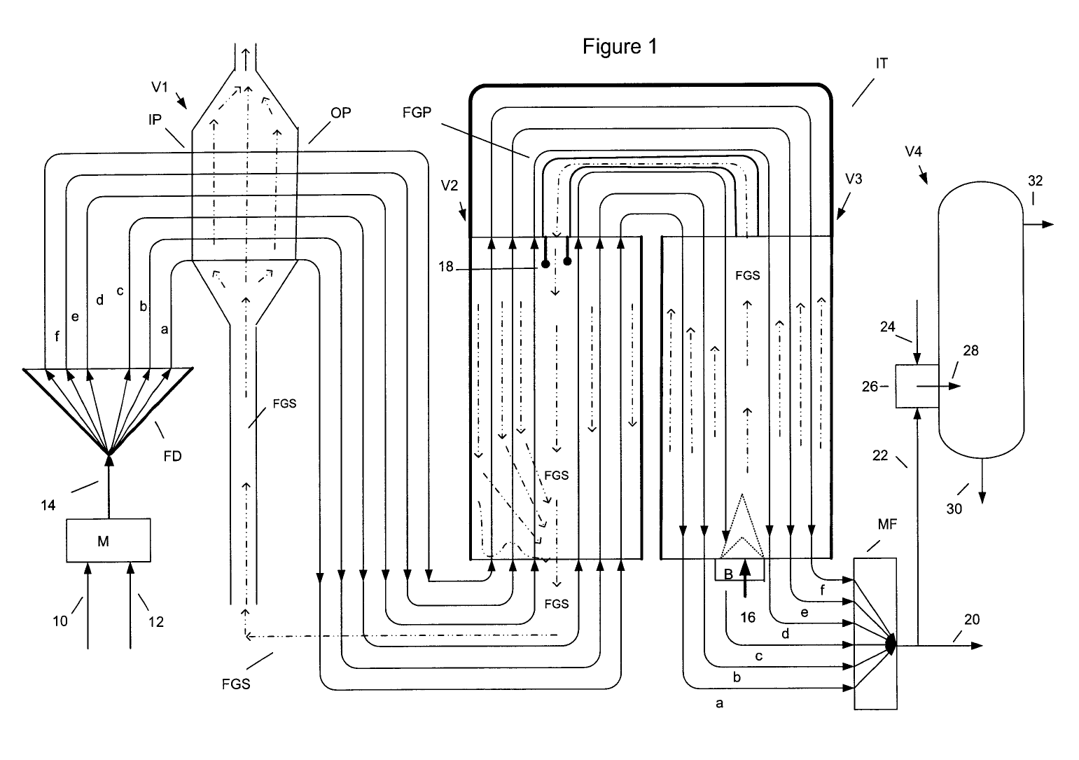

This invention can be better understood with reference to the figures hereof. The present process utilizes a stream reformer comprised of a plurality of serially connected temperature stages, each at a higher temperature than the previous upstream temperature stage. At least three such temperature stages are used, but a fourth temperature stage can also be added for feedstocks that require temperatures greater than about 1750° F. FIG. 1 hereof is a simplified schematic of a preferred reformer used in the practice of the present invention. The reformed is comprised of a flow divider FD which is capable of dividing a feedstream into any number of predetermined streams. For the purpose of this invention the feedstream will be divided into six substantially equal streams. Flow dividers are well known to those having at least ordinary skill in the art and thus there is no need to discuss them in detail herein. Each stream is conducted via feed tubes a-f to inlet ports IP on the side of the reactor vessel VI. It is preferred that all reactor vessels of this invention be cylindrical in shape, although other suitable shapes could also be used. All construction materials, including reactor vessels and reactor tubes through which the feedstock passes through the four stages are manufactured from high temperature alloys suitable for use at the temperatures and conditions of the particular reactor vessel in which they are located. It is preferred that the reaction vessels of the present invention be insulated, more preferably in its interior with a suitable insulating material. It is also preferred that the reactor tubes be cast tubes comprised of a high temperature alloy. It has been found by the inventor hereof that cast alloy feed tubes are better able to withstand the reaction conditions of the reactor vessels of the present invention better than extruded or rolled tubes. Therefore, cast feed tubes are preferred.

The divided feedstreams are transported through reactor vessel VI through reactor tubes a-f and are fluidly connected to outlet ports OP which are in turn fluidly connected to feed tubes within the interior of reactor vessel V1. In fact, all individual feed tubes are fluidly connected from the flow divider FD to manifold MF so that the feedstream passes serially through all reaction vessels. Reactor vessel V1 is preferably a shell and tube type vessel and will be run during the stream reforming reaction at a temperature from about 650° F. to about 800° F. The heat used to run reactor vessel V1 is derived from flue gas stream FGS that originates in stage 3 reactor vessel V3 by burner B which is fueled via line 16 preferably with natural gas, or the synthesis gas produced by the process of the present invention. Feed tubes exit reactor vessel V1 at outlet ports OP and are fluidly connected to inlet ports at the bottom of stage 2 reactor vessel V2 which are fluidly connected to a plurality of feed tubes extending vertically throughout the length of reactor vessel V2 and ending at outlet ports at the top of reactor vessel V2. Reactor vessel V2 will be operated in the temperature range of about 1300° F. to about 1450° F. The heat to run reactor vessel V2 is also obtained from the flue gas stream FGS produced by burner B located at the bottom of reactor vessel V3. In the event flue gas stream FGS does not provide an adequate amount of heat to maintain reactor vessel at a temperature from about 1300° F. to about 1450° F. trim burner 18 may be used to add heat to flue gas stream FGS. It is preferred that trim burner 18 also be fueled by use of natural gas or a portion of the product synthesis gas. It is also preferred that the trim burner be an annular shaped burner surrounding the perimeter of the opening of flue gas pipe FGP which is fluidly connected to the top of reactor vessel V2 to receive flue gas from reactor vessel V3.

The reaction product of reactor vessel V2 continues flowing downstream through a plurality of feed tubes that fluidly connect vertically oriented feed tubes in reactor vessel V2 and the plurality of feed tubes vertically oriented in reactor vessel V3. Reactor vessel V3 will be operated at a temperature in the range of about 1450° F. to about 1750° F. where further reaction of the hydrocarbons in the reaction product from V2 takes place. An insulating top, or cover, IT is provided that encloses the reaction tubes and the tops of reactor vessels V2 and V3 to prevent an undesirable amount of heat loss from feed tubes extending from reactor vessel V2 to V3. The tubular members exit the bottom the reactor vessel V3 and into manifold MF where the reaction product streams are combined into one stream which exits manifold MF via line 20. If a feedstock, such as natural gas or methanol, is used and the steam reforming reaction is completed in reactor vessel V3, the product synthesis gas can be collected and stored or sent for further downstream processing. If the feedstock is relatively refractory and contains a high carbon content that requires additional high temperature reforming, then the reaction product exiting manifold MF is sent via line 22 to a fourth stage reactor vessel V4 by first conducting it to a mixer 26 where it is mixed with an effective amount of an oxygen-containing gas, preferably substantially pure oxygen via line 24. It will be understood that mixer 26 can be either external or internal to reactor vessel V4. It is preferred that it be external. The mixture of reaction product from reactor vessel V3 and oxygen-containing gas enter reactor vessel V4 at 28 where it further combusts at temperatures from about 1750° F. to about 2100° F., preferably at a temperature from about 1800° F. to about 2000° F. The final reaction product synthesis gas exits the steam reformer at outlet 32 and is collected and stored, or transported off site, or passed to a downstream process unit for further processing.

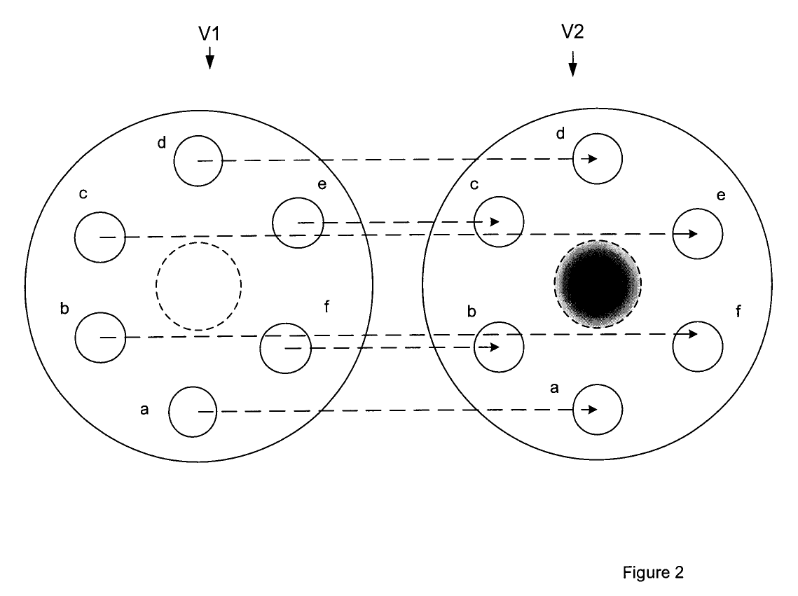

FIG. 2 hereof is a cross-sectional view along A-A of FIG. 1 hereof showing base plates BP2 and BP3 for reactor vessels V2 and V3 respectively. Also shown respectively for each reactor vessels V2 and V3 are outside walls W2 and W3 and tubular members a, b, c, d, e, and f. Tubular members a, b, c, d, e, and f extend vertically upward from base plate BP2 and over to reactor vessel V3 where they extend vertically downward to based plate BP3. FL is the flame from burner B.

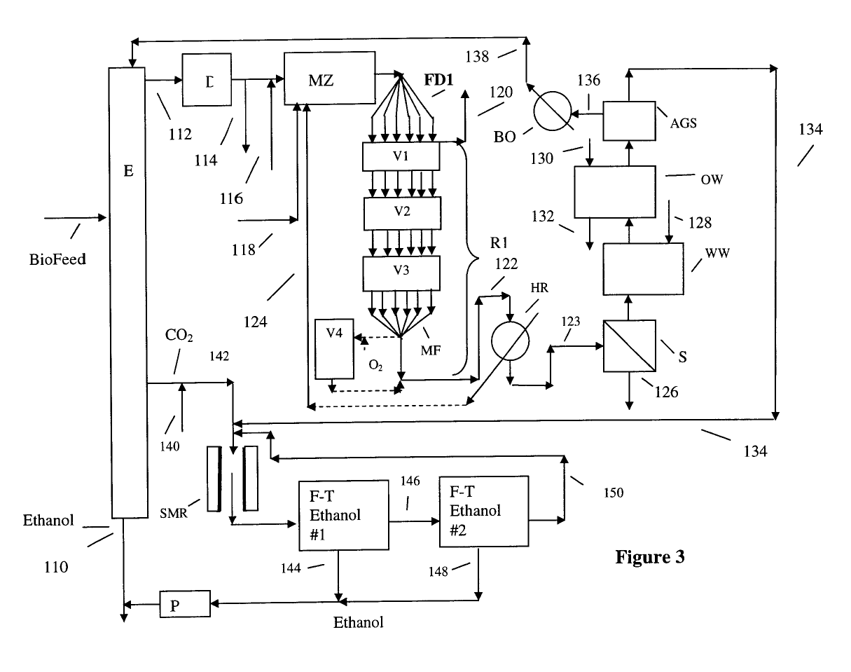

Turning now to FIG. 3 hereof, the biomass feedstock, BioFeed, preferably corn or blackstrap molasses, is fed to an ethanol processing plant E wherein an ethanol stream, carbon dioxide, and distiller grains are produced. Such processing plants are well known in the art and thus there is no need to present a detailed technical discussion of these plants in this document. The ethanol is collected via line 110 and the distillers grains, DG can be sent to drier D via line 112 where they are dried and either collected for sale as livestock feed via line 114 or as a waste by-product and used a fuel to a boiler. If the distillers grains have little or no value as a livestock feed and are sent to further processing then they can be sent either wet or dry to mixing zone MZ. If there is excess waste heat available then it is preferred that they be dried in drier D. If not they can be sent directly to the mixing zone MZ. If they are sent to mixing zone MZ they are mixed with an effective amount of steam, preferably superheated steam, via line 118 and optionally an additional biomass, preferably corn stover or a cellulosic material, such as wood via line 116. In cases where the distiller grains have value as a livestock feed then little or no distillers grains will be passed to mixing zone MZ and only the additional biomass material will be used as the feedstock to mixing zone MZ. In cases where the distillers grains have little or no value as livestock feed additional biomass material may not be needed to produce the synthetic fuel gas as fuel for the boiler. The resulting mixture from mixing zone MZ is passed to reforming zone RI which is comprised of at least three, and optionally four, serially connected temperature stages conducted in vessels V1, V2, V3, and V4.

The steam, preferably superheated steam, which will be at a temperature from about 315° C. to about 700° C. acts as both a source of hydrogen as well as a transport medium. The amount of superheated steam to feedstock will be an effective amount. By effective amount we mean at least that amount needed to provide sufficient transport of the feedstock through the reaction tubes. That ratio of superheated steam to feedstock, on a volume to volume basis, will typically be from about 0.2 to 2.5, preferably from about 0.3 to 1.0. The temperature conditions for the reforming stages will be described later in detail. Fluidization will typically result and can realize fluid reforming by virtue of good contact among steam, feed polymers and heat decomposition products of feedstock liberated in the gas phase.

The mixture of steam and feedstock, which will be at a temperature of above its dew point of greater than about 230° C., is fed to stream reformer R1 through a flow divider FD where it is distributed into the plurality of reactor tubes of effective internal diameter and length within a metal cylindrical vessel of suitable size. Flow divider FD can be any suitable design that will divide the feedstock substantially equally among the plurality of reactor tubes. The temperature of the mixture entering first reformer vessel V1 will be at least about 230° C. Reactor vessel V1 is preferably a shell and tube type vessel and will be run during the stream reforming reaction at a temperature from about 650° F. to about 800° F. The heat used to run reactor vessel V1 is shown in FIG. 1 hereof and is derived from flue gas stream FGS that originates in stage 3 reactor vessel V3 that contains a burner that is fueled via line 16 preferably with natural gas, or a portion of the synthesis gas produced by conduction steam reforming in the apparatus of the present invention. Feed tubes exit reactor vessel V1 and are fluidly connected to reaction tubes contained within stage 2 reactor vessel V2. Reactor vessel V2 will be operated in the temperature range of about 1300° F. to about 1450° F. The heat to run reactor vessel V2 is also obtained from the flue gas stream produced by a burner located at the bottom of reactor vessel V3 (see FIG. 1 hereof).

The reaction product of reactor vessel V2 continues flowing downstream through a plurality of feed tubes that fluidly connect vertically oriented feed tubes in reactor vessel V2 and the plurality of feed tubes, preferably vertically oriented in reactor vessel V3. Reactor vessel V3 is operated at a temperature in the range of about 1450° F. to about 1750° F. where further reaction of the hydrocarbons in the reaction product from V2 takes place. The tubular members will exit reactor vessel V3 and into a manifold where the reaction product streams are combined into one stream. If a feedstock is used that requires additional steam reforming at temperatures higher than that of stage 3 then it is sent to a fourth stage, reaction vessel V4 along with an effective amount of oxygen-containing gas. If the intended reaction is complete in stage 3 then the resulting gaseous product is sent from reaction vessel V3 to manifold MF. The fourth stage, if used is operated at temperatures from about 1750° F to about 2100° F., preferably at a temperature from about 1800° F. to about 2000° F.

The flue gas is exhausted from the reformer via line 120 and the product syngas stream from reformer R1 is conducted via line 122 to heat recovery zone HR where it is preferred that water be the heat exchange medium and that the water be passed as preheated steam to reformer R1 via line 124 where it is further heated to produce at least a portion of the superheated steam introduced into mixing zone MZ. Heat Recovery zone HR can be any suitable heat exchange device, such as the shell-and-tube type wherein water is used to remove heat from product stream 122. From heat recovery zone HR the product syngas is passed, via line 123, to separation zone S which contains a gas filtering means and preferably a cyclone (not shown) and optionally a bag house (not shown) to remove at least a portion, preferably substantially all, of the remaining ash and other solid fines from the syngas. The filtered solids are collected via line 126 for disposal.

The filtered syngas stream is then passed to water wash zone WW wherein it is conducted upward and countercurrent to down-flowing water via line 128. The water wash zone preferably comprises a column packed with conventional packing material, such as copper tubing, pall rings, metal mesh or other such materials. The syngas passes upward countercurrent to down-flowing water which serves to further cool the syngas stream to about ambient temperature, and to remove any remaining ash that may not have been removed in separation zone S. The water washed syngas stream is then passed to oil wash zone OW where it is passed countercurrent to a down-flowing organic liquid stream, via line 130, to remove any organics present, such as benzene, toluene, xylene, or heavier hydrocarbon components via line 132 that may have been produced in the reformer. The down-flowing organic stream will be any organic stream in which the organic material being removed is substantially soluble. It is preferred that the down-flowing organic stream be a hydrocarbon stream, more preferably a petroleum fraction. The preferred petroleum fractions are those boiling in naphtha to distillate boiling range, more preferably a C₁₆ to C₂₀ hydrocarbon stream, most preferably a C₁₈ hydrocarbon stream.

The resulting syngas stream is conducted to acid gas scrubbing zone AGS wherein acidic gases are removed. Although the acid gases CO₂ and H₂S are typically removed from stream with use of an acid gas scrubbing zone the syngas fuel stream resulting from reforming distillers grains will not contain sulfur, thus only the acid gas CO₂ will be removed. The CO₂ is removed via line 134 and sent stream methane reformer SMR along with the CO₂ stream from the ethanol plant via line 140. Any suitable acid gas treating technology can be used in the practice of the present invention. Also, any suitable scrubbing agent, preferably a basic solution can be used in the acid gas scrubbing zone AGS that will adsorb the desired level of acid gases from the vapor stream. If the methane product stream is to be used for the production of methanol, then at least that stoichiometric amount of CO₂ needed to result in the production of syn gas. One suitable acid gas scrubbing technology is the use of an amine scrubber. Non-limiting examples of such basic solutions are the amines, preferably diethanol amine, mono-ethanol amine, and the like. More preferred is diethanol amine. Another preferred acid gas scrubbing technology is the so-called “Rectisol Wash” which uses an organic solvent, typically methanol, at subzero temperatures. The scrubbed stream can also be passed through one or more guard beds (not shown) to remove any trace amounts of catalyst poisoning impurities such as sulfur, halides etc.

The resulting synthetic gaseous fuel product exits acid gas scrubbing zone AGS via line 136 and is used as fuel to run boiler BO for producing steam to be sent to fermentation zone E via line 138. The other product from the fermentation zone is carbon dioxide which is used to make additional ethanol.

CO₂ from the ethanol plant E and CO₂ from the acid gas scrubbing zone AGS are combined and mixed with a hydrocarbon, preferably natural gas (methane) via line 140, which mixture is conducted via line 142 to steam methane reformer SMR which is a conventional catalytic methane reformer well known in the art. Steam reforming of natural gas, sometimes referred to as steam methane reforming (SMR) is the most common method of producing commercial bulk hydrogen. One preferred type of steam methane reformer suitable for use herein is the type taught by Haldor Topsoe in U.S. Pat. No. 5,932,141 which is incorporated herein by reference. Steam methane reformers are typically operated at temperatures from about 700° C. to about 1100° C. in the presence of a metal-based catalyst, typically a nickel-based catalyst filled tubes where steam reacts with methane to yield carbon monoxide and hydrogen. Thus, the synthetic gaseous product from the steam methane reformer of the present invention will be comprised primarily of hydrogen with lesser amounts of carbon monoxide and carbon dioxide.

The synthetic gaseous product is conducted to an ethanol Fischer-Tropsch reactor which is catalyzed to favor the production of ethanol. Any conventional ethanol producing catalyst can be used in the Fischer-Tropsch reactor of the present invention. Preferred catalysts are those that are based on cobalt with contain other elements selected from the group consisting of manganese, zinc, chromium and/or molybdenum, aluminum, and an alkali or alkaline earth metal promoter, with potassium carbonate being preferred for economic reasons. The more preferred ethanol catalysts will be comprised of about 65 wt. % to about 75 wt. % cobalt, about 4 wt. % to about 12 wt. % manganese, about 4 wt. % to about 10 wt. % zinc, about 6 wt. % to about 10 wt. % chromium, up to about 20 wt. % Mo, and/or about 6 wt. % to about 10 wt. % aluminum, wherein all weight percents are based only on the metal content without binder or carrier.

While the catalyst as used consists primarily of the above elements in their elemental form, the catalysts are typically prepared from a mixture of metal salts. Nitrates and carbonates are preferred. The catalysts used in the both the methanol reactor and the ethanol reactor of this invention will be subjected to a “conditioning” process wherein the salts are largely reduced to their metallic state, with some oxides remaining to form a lattice structure referred to as spinels. The spinel structure help give the catalysts their overall special structure. The catalysts may be used in their pure (or concentrated) form, or they may be diluted with carbon, by loading onto carbon pellets. The later is often referred to as supported catalyst. A “pure” catalyst will tend to run hotter than a supported catalyst. On the other hand a “pure” catalyst will be more active and hence can be used at lower reaction temperatures. Thus a compromise must often be reached between the desirability of using a more reactive catalyst and the need to dilute it in order to facilitate temperature control.

The temperature range in the Fischer-Tropsch reaction zone will be from about 240° C. to about 420° C. at a gas hourly space velocity of about 8,000 per hour to about 50,000 per hour and at a total pressure of about 100 psig to about 3000 psig. It is preferred that the ethanol reactors be comprised of a series of stainless steel reactor tubes, each of suitable diameter, for example from about 1 to 2 inches in diameter. The reactor tubes are loaded with a suitable catalyst that favors the production of ethanol. Larger diameter reactor tubes give higher production capacity, but also allow for the generation of higher temperatures that is deleterious to the catalyst. Thus, the tube diameter will usually be selected as a compromise between flow through capacity desired and the ease with which the heat can be controlled.

The reaction products from Fischer-Tropsch ethanol reactor #1 will be a predominantly ethanol stream and a purge gas stream comprised primarily of unreacted synthesis feed gas (methane and CO₂). The predominantly ethanol stream is sent, via line 144, to purification zone P where it is purified. The ethanol stream will typically contain an unacceptable amount of water and purification zone will preferably be fraction distillation zone that will result in at least a 95.6% ethanol/4.4% water azeotrope mixture. If further purification is needed such techniques as drying using hexane, benzene, or other azeotrope liquids, or the use of a molecular sieve or membrane can be used. The purified ethanol is collected and added to ethanol from the ethanol plant E. The purge gas stream is conducted, via line 146, to a second Fischer-Tropsch ethanol reactor #2 which is run within the conditions and with a catalyst as described above. The resulting ethanol stream from this second Fischer-Tropsch reactor will also be sent, via line 148, to purification zone P to purify the ethanol stream to the desired level of purity. The resulting purge gas stream from this second Fischer-Tropsch will be recycled via line 150 to steam methane reformer SMR.

What is claimed is:

1. A process for converting biomass to ethanol, which process comprising:
a) milling biomass to produce a powdered meal;
b) adding an effective amount of water and a first enzyme to the meal to liquefy the meal thereby producing a mash;
c) cooking the mash for an effective amount of time at an effective temperatures in the presence of a second enzyme;
d) adding an effective amount of yeast to said cooked mash and passing said cooked mash containing said yeast to a fermentation zone;
e) fermentating said cooked mash containing said yeast for an effective amount of time thereby producing a first ethanol product stream, a carbon dioxide stream, and distiller grains;
f) distilling off the ethanol product;
g) collecting the ethanol product;
h) separating the carbon dioxide from the distiller grains;
i) drying said distiller grains;
j) determining if said distiller grains are suitable as a livestock feed;
k) collecting the distiller grains if the distiller grains are not suitable as a livestock feed;
l) conducting an effective amount of steam and at least a portion of the dried distiller grains to a mixing zone and forming a mixture to a steam reforming zone comprised of at least three serially connected reforming stages, each operated at progressively higher temperature and wherein the temperature of the first reforming stage is from about 650° F. to about 800° F. and wherein a synthetic gaseous product stream is produced;
m) conducting an effective amount of a biomass co-feed to said mixing zone in the event the amount of distillers grains is insufficient for a predetermined feed rate to said reforming zone;
n) passing said synthetic gaseous product stream from said steam reforming zone to a heat recovery zone wherein the temperature of said synthetic gaseous product stream is substantially lowered and wherein steam is generated;
o) passing said lowered temperature synthetic gaseous product stream to a solids recovery zone wherein substantially any solids present are removed;
p) passing said synthetic gaseous product stream from said solids recovery zone to an organics removal zone wherein substantially any remaining organic material is removed by contact with an organic liquid in which the organic material is at least partially soluble;
q) passing said synthetic gaseous product stream from said organics removal zone to a acid gas removal zone wherein CO₂ is removed, thereby resulting in an acid gas depleted synthetic gaseous product stream and a CO₂ stream;
r) passing said acid gas depleted synthetic gaseous product stream as fuel to a boiler that produces steam;
s) passing at least a portion of said steam produced in s) above to the fermentation zone;
t) passing at least a portion of the carbon dioxide streams from both the fermentation zone and the acid gas removal zone, along with an effective amount of methane, to a catalytic steam reformer;
u) reforming said mixture of carbon dioxide and methane in the presence of a stream reforming catalyst at temperatures from about 800° C. to about 1000° C. thereby resulting in a synthesis gas product stream, which synthesis gas product stream is comprised of hydrogen, carbon oxides, and methane
v) passing said synthesis gas product stream to a first Fischer-Tropsch ethanol reactor wherein it is reacted in the presence of a catalyst capable of converting a portion of the synthesis gas to ethanol wherein there is produced a second ethanol product stream, steam, and a first purge gas stream comprised of unreacted synthesis gas;
w) passing said second ethanol product stream to a purification zone wherein impurities are removed form the ethanol;
x) passing said steam and purge gas stream from step v) to a second Fischer Tropsch ethanol reactor wherein a third ethanol product stream is produced steam, and a second purge gas stream comprised of unreacted synthesis gas;
y) passing said third ethanol product stream to a purification zone wherein impurities are removed;
z) passing at least a portion of said steam and second purge gas stream from step x) to said catalytic steam reformer.

2. The process of claim 1 wherein the biomass is selected from the group consisting of corn and a sugar substance.

3. The process of claim 1 wherein the biomass is corn.

4. The process of claim 3 wherein the temperature of the second reforming stage is from about 1450° F. to about 1750° F.

5. The process of claim 4 wherein the temperature of the third reforming stage is from about 1750° F. to about 2100° F.

6. The process of claim 1 wherein the biomass co-feed is used in step m).

7. The process of claim 3 wherein the first enzyme is alpha-amylase.

8. The process of claim 3 wherein the second enzyme is glucoamylase.
